(19) United States
(12) Patent Application Publication
(10) Pub. No.: US 2026/0176220 A1
(43) Pub. Date: Jun. 25, 2026

(54) METHODS FOR FORMING DEHYDROGENATED PRODUCTS UTILIZING COMBUSTION BYPASS OF SOME CATALYST

(71) Applicant: Dow Global Technologies LLC, Midland, MI (US)

(72) Inventors: Davy Nieskens, Terneuzen (NL); Matthew T. Pretz, Lake Jackson, TX (US); Brian W. Goodfellow, Sugarland, TX (US); Andrzej Malek, Midland, MI (US)

(73) Assignee: Dow Global Technologies LLC, Midland, MI (US)

(21) Appl. No.: 19/128,410

(22) PCT Filed: Nov. 22, 2023
(86) PCT No.: PCT/US2023/080921

§ 371 Date: May 8, 2025

Related U.S. Application Data
(60) Provisional application No. 63/428,520, filed on Nov. 29, 2022.

(51) Int. Cl. C07C 5/333; B01J 8/00

(52) U.S. Cl. CPC C07C 5/3337; B01J 8/003; B01J 8/0055; B01J 2523/41

(57) ABSTRACT

A dehydrogenated product may be formed by a method that includes reacting a feed stream in the presence of a catalyst by a dehydrogenation reaction in a reactor to form a dehydrogenated product, separating at least a portion of the product stream from the catalyst, separating the catalyst into at least a first catalyst portion and a second catalyst portion, and passing the first catalyst portion to a combustor. The first catalyst portion may be heated in the combustor by the combustion of supplemental fuel. The method may further include passing the first catalyst portion out of the combustor and combining the first catalyst portion with the second catalyst portion downstream of the combustor to form a recombined catalyst stream, such that the second catalyst portion bypasses the combustor. The method may further include passing the recombined catalyst stream through an oxygen treatment zone, and passing the recombined catalyst stream to the reactor.

### CROSS-REFERENCE TO RELATED APPLICATIONS

This application claims the benefit of U.S. Provisional Application Ser. No. 63/428,520 filed Nov. 29, 2022, the entire disclosure of which is hereby incorporated herein by reference.

### TECHNICAL FIELD

Embodiments described herein generally relate to chemical processing and, more specifically, to methods and systems for light olefin production.

### BACKGROUND

Light olefins, such and propylene, may be used as base materials to produce many different materials, such as polypropylene, which may be used in packaging, construction, and textiles. As a result of this utility, there is a worldwide demand for light olefins. Suitable processes for producing light olefins generally depend on the given chemical feed and include those that utilize fluidized catalysts. For example, light olefins may be formed by the catalytic dehydrogenation of alkanes. However, there is a need for improvement in the systems used to make dehydrogenated products.

### SUMMARY

Olefinic products may be produced by catalytic dehydrogenation, where a catalyst is circulated between a reactor (where the dehydrogenation reaction take place) and a combustor (where the catalyst is heated by combustion of a supplemental fuel) followed by exposure to oxygen in an oxygen treatment zone. According to embodiments described herein, a portion of the catalyst may bypass the combustor and be recombined with the portion of the catalyst that passes to the combustor downstream of the combustor and upstream of the oxygen treatment zone. Such a process may be beneficial by improving catalyst lifecycle time and reduce catalyst deactivation that may occur when exposed to combustion of supplemental fuels in the combustor.

According to one or more embodiments of the present disclosure, a dehydrogenated product may be formed by a method comprising reacting a feed stream in the presence of a catalyst by a dehydrogenation reaction in a reactor to form a dehydrogenated product, separating at least a portion of the product stream from the catalyst, separating the catalyst into at least a first catalyst portion and a second catalyst portion, and passing the first catalyst portion to a combustor. The first catalyst portion may be heated in the combustor by the combustion of supplemental fuel. The method may further comprise passing the first catalyst portion out of the combustor and combining the first catalyst portion with the second catalyst portion downstream of the combustor to form a recombined catalyst stream, such that the second catalyst portion bypasses the combustor. The method may further comprise passing the recombined catalyst stream through an oxygen treatment zone, and passing the recombined catalyst stream to the reactor.

It is to be understood that both the preceding general description and the following detailed description describe various embodiments and are intended to provide an overview or framework for understanding the nature and character of the claimed subject matter. Additional features and advantages of the embodiments will be set forth in the detailed description and, in part, will be readily apparent to persons of ordinary skill in the art from that description, which includes the accompanying drawing and claims, or recognized by practicing the described embodiments. The drawing is included to provide a further understanding of the embodiments and, together with the detailed description, serves to explain the principles and operations of the claimed subject matter. However, the embodiment depicted in the drawing is illustrative and exemplary in nature, and not intended to limit the claimed subject matter.

### BRIEF DESCRIPTION OF THE DRAWING

The following detailed description may be better understood when read in conjunction with the following drawing, in which:

FIG. 1 schematically depicts a reactor system, according to one or more embodiments of the present disclosure;

FIG. 2 shows experimental data, as is described herein; and

FIG. 3 shows additional experimental data, as is described herein.

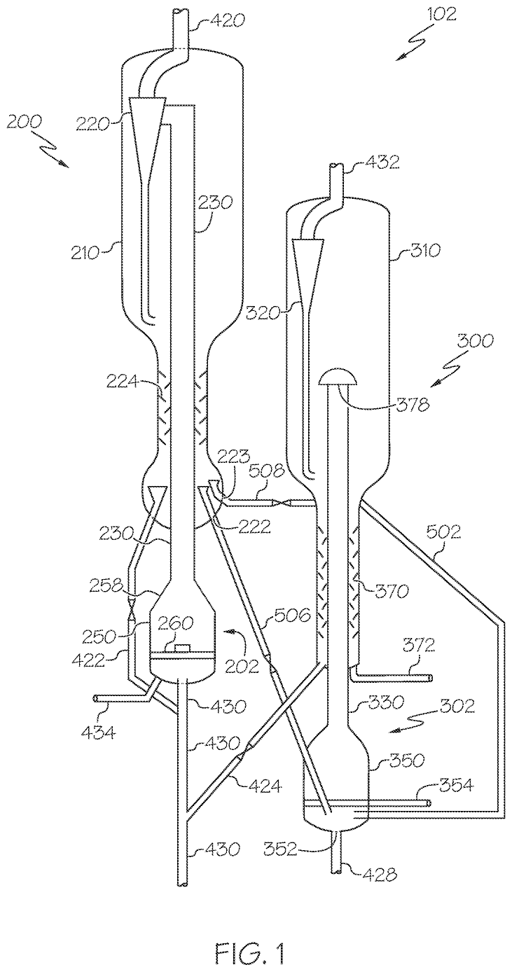

When describing the simplified schematic illustration of FIG. 1, the numerous valves, temperature sensors, electronic controllers, and the like, which may be used and are well known to a person of ordinary skill in the art, are not included. Further, accompanying components that are often included in such reactor systems, such as air supplies, heat exchangers, surge tanks, and the like are also not included. However, it should be understood that these components are within the scope of the present disclosure.

Reference will now be made in greater detail to various embodiments, some of which are illustrated in the accompanying drawing.

### DETAILED DESCRIPTION

The present disclosure is directed to methods and systems for making chemical products, such as light olefins, by dehydrogenation, as is described herein. The presently disclosed embodiments may utilize reactor systems that include reactors (where dehydrogenation reaction is performed) and combustors (where the catalyst is regenerated). As is described herein in detail, the catalyst may cycle between these two units. Described herein are systems and methods that allow for a portion of the catalyst to bypass the combustor, better understood with respect to the embodiments described herein, such as that using the context of FIG. 1.

Embodiments presently disclosed are described in detail herein in the context of the reactor system of FIG. 1 operating as a fluidized dehydrogenation reactor system to produce light olefins, such as propylene. However, it should be understood that the principles disclosed and taught herein may be applicable to other systems which utilize different system components oriented in different ways. For example, the concepts described may be equally applied to other systems with alternate reactor units and combustors (sometimes called regeneration units herein), such as those that operate under non-fluidized conditions or include downers rather than risers. It should be further understood that not all portions of FIG. 1 should be construed as essential to the claimed subject matter.

Now referring to FIG. 1, an example reactor system 102 that may be suitable for use with the methods and/or apparatuses described herein is schematically depicted. The reactor system 102 generally comprises multiple system components, such as a reactor portion 200 and a catalyst processing portion 300. As described herein, “system components” refer to portions of the reactor system 102, such as reactors, separators, transfer lines, combinations thereof, and the like. As used herein in the context of FIG. 1, the reactor portion 200 generally refers to the portion of a reactor system 102 in which the major process reaction takes place (e.g., dehydrogenation) to form the product stream. A feed stream enters the reactor portion 200, is converted to a product stream (containing product and unreacted feed), and exits the reactor portion 200. The reactor portion 200 comprises a reactor 202 which may include an upstream reactor section 250 and a downstream reactor section 230. According to one or more embodiments, as depicted in FIG. 1, the reactor portion 200 may additionally include a catalyst separation section 210, which serves to separate the catalyst from the chemical products formed in the reactor 202. Also, as used herein, the catalyst processing portion 300 generally refers to the portion of the reactor system 102 where the catalyst is in some way processed, such as by combustion, to, e.g., improve catalytic activity by decoking and/or heating the catalyst. The catalyst processing portion 300 may comprise a combustor 350 and a riser 330, and may additionally comprise a catalyst separation section 310. In one or more embodiments, the catalyst separation section 210 may be in fluid communication with the combustor 350 (e.g., via standpipe 426) and the catalyst separation section 310 may be in fluid communication with the upstream reactor section 250 (e.g., via standpipe 424 and transport riser 430).

Generally as is described herein, in embodiments illustrated in FIG. 1, catalyst is cycled between the reactor portion 200 and the catalyst processing portion 300. It should be understood that when “catalysts” are referred to herein, they may refer to solid materials that are catalytically active for a desired reaction, or may equally refer to other particulate solids referenced with respect to the system of FIG. 1 which do not necessarily have catalytic activity but affect the reaction. The terms “catalytic activity” and “catalyst activity” refer to the degree to which the catalyst is able to catalyze the reactions conducted in the reactor system. The catalyst that exits the reactor portion 200 may be deactivated catalyst. As used herein, “deactivated” may refer to a catalyst which has reduced catalytic activity or is cooler as compared to catalyst entering the reactor portion 200. However, deactivated catalyst may maintain some catalytic activity. Reduced catalytic activity may result from contamination with a substance such as coke. Reactivation (sometimes called “regeneration” herein) may remove the contaminant such as coke, raise the temperature of the catalyst, or both. In embodiments, deactivated catalyst may be reactivated by catalyst reactivation in the catalyst processing portion 300. The deactivated catalyst may be reactivated by, but not limited to, removing coke by combustion, recovering catalyst acidity, oxidizing the catalyst, other reactivation process, or combinations thereof. In some embodiments, the catalyst may be heated during reactivation by combustion of a supplemental fuel, such as hydrogen, methane, ethane, propane, natural gas, or combinations thereof. The reactivated catalyst from the catalyst processing portion 300 is then passed back to the reactor portion 200.

In non-limiting examples, the reactor system 102 described herein may be utilized to produce light olefins from hydrocarbon feed streams. According to one or more embodiments, the reaction may be a dehydrogenation reaction. According to such embodiments, the hydrocarbon feed stream may comprise one or more of ethyl benzene, ethane, propane, n-butane, and i-butane. In one or more embodiments, the hydrocarbon feed stream may comprise at least 50 wt. %, at least 60 wt. %, at least 70 wt. %, at least 80 wt. %, at least 90 wt. %, at least 95 wt. % or even at least 99wt. % of ethyl benzene. In one or more embodiments, the hydrocarbon feed stream may comprise at least 50 wt. %, at least 60 wt. %, at least 70 wt. %, at least 80 wt. %, at least 90 wt. %, at least 95wt. % or even at least 99 wt. % of ethane. In additional embodiments, the hydrocarbon feed stream may comprise at least 50 wt. %, at least 60 wt. %, at least 70 wt. %, at least 80 wt. %, at least 90wt. %, at least 95 wt. % or even at least 99 wt. % of propane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of n-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of the sum of ethane, propane, n-butane, and i-butane.

As described with respect to FIG. 1, the feed stream may enter feed inlet 434 into the reactor 202, and the product stream may exit the reactor system 102 via pipe 420. According to one or more embodiments, the reactor system 102 may be operated by feeding a chemical feed (e.g., in a feed stream) and a fluidized catalyst into the upstream reactor section 250. The chemical feed contacts the catalyst in the upstream reactor section 250, and each flow upwardly into and through the downstream reactor section 230 to produce a chemical product.

Now referring to FIG. 1 in detail, the reactor portion 200 may comprise an upstream reactor section 250, a transition section 258, and a downstream reactor section 230, such as a riser. The transition section 258 may connect the upstream reactor section 250 with the downstream reactor section 230. As depicted in FIG. 1, the upstream reactor section 250 may be positioned below the downstream reactor section 230. Such a configuration may be referred to as an upflow configuration in the reactor 202. The upstream reactor section 250 may include a vessel, drum, barrel, vat, or other container suitable for a given chemical reaction. As depicted in FIG. 1, the upstream reactor section 250 may be connected to the downstream reactor section 230 via the transition section 258. The upstream reactor section 250 may generally comprise a greater cross-sectional area than the downstream reactor section 230. The transition section 258 may be tapered from the size of the cross-section of the upstream reactor section 250 to the size of the cross-section of the downstream reactor section 230 such that the transition section 258 projects inwardly from the upstream reactor section 250 to the downstream reactor section 230. For example, the transition section 258 may be a frustum.

The upstream reactor section 250 may be connected to a transport riser 430, which, in operation may provide reactivated catalyst in a feed stream to the reactor portion 200. The reactivated catalyst and/or reactant chemicals may be mixed with a distributor 260 housed in the upstream reactor section 250. The catalyst entering the upstream reactor section 250 via transport riser 430 may be passed through standpipe 424 to a transport riser 430, thus arriving from the catalyst processing portion 300. In some embodiments, catalyst may come directly from the catalyst separation section 210 via standpipe 422 and into a transport riser 430, where it enters the upstream reactor section 250, where in such embodiments some of the catalyst is not passed through the catalyst processing portion 300. The catalyst can also be fed via standpipe 422 directly to the upstream reactor section 250 (not depicted in FIG. 1). This catalyst may be somewhat deactivated, but may still, in some embodiments, be suitable for reaction in the upstream reactor section 250, particularly when used in combination with reactivated catalyst.

Still referring to FIG. 1, in one or more embodiments, based on the shape, size, and other processing conditions (such as temperature and pressure) in the upstream reactor section 250 and the downstream reactor section 230, the upstream reactor section 250 may operate as a fluidized bed, such as in a fast fluidized, turbulent, or bubbling bed upflow reactor, while the downstream reactor section 230 may operate in more of a plug flow manner, such as in a riser reactor. For example, the reactor 202 of FIG. 1 may comprise an upstream reactor section 250 operating as a fast fluidized, turbulent, or bubbling bed reactor and a downstream reactor section 230 operating as a dilute phase riser reactor, with the result that the average catalyst and gas flow moves concurrently upward. As the term is used herein, “average flow” refers to the net flow, i.e., the total upward flow minus the retrograde or reverse flow, as is typical of the behavior of fluidized particles in general. As described herein, a “fast fluidized” reactor may refer to a reactor utilizing a fluidization regime wherein the superficial velocity of the gas phase is greater than the choking velocity and may be semi-dense in operation. As described herein, a “turbulent” reactor may refer to a fluidization regime where the superficial velocity of less than the choking velocity and is more dense than the fast fluidized regime. As described herein, a “bubbling bed” reactor may refer to a fluidization regime wherein well defined bubbles in a highly dense bed are present in two distinct phases. The “choking velocity” refers to the minimum velocity required to maintain solids in the dilute-phase mode in a vertical conveying line. As described herein, a “dilute phase riser” may refer to a riser reactor operating at transport velocity, where the gas and catalyst have about the same velocity in a dilute phase.

According to embodiments, the chemical product and the catalyst may be passed out of the downstream reactor section 230 to a separation device 220 in the catalyst separation section 210, where the catalyst is separated from the chemical product, which is transported out of the catalyst separation section 210. According to one or more embodiments, following separation from vapors in the separation device 220, the catalyst may generally move through the stripper 224 to the catalyst outlet port 222 where the catalyst is transferred out of the reactor portion 200 via standpipe 426 and into the catalyst processing portion 300.

According to one or more embodiments, the separation device 220 may be a cyclonic separation system, which may include two or more stages of cyclonic separation. In embodiments where the separation device 220 comprises more than one cyclonic separation stages, the first separation device into which the fluidized stream enters is referred to a primary cyclonic separation device. The fluidized effluent from the primary cyclonic separation device may enter into a secondary cyclonic separation device for further separation. Primary cyclonic separation devices may include, for example, primary cyclones, and systems commercially available under the names VSS (commercially available from UOP), LD2 (commercially available from Stone and Webster), and RS2 (commercially available from Stone and Webster). Primary cyclones are described, for example, in U.S. Pat. Nos. 4,579,716; 5,190,650; and 5,275,641, which are each incorporated by reference in their entirety herein. In some separation systems utilizing primary cyclones as the primary cyclonic separation device, one or more set of additional cyclones, e.g. secondary cyclones and tertiary cyclones, are employed for further separation of the catalyst from the product gas. It should be understood that any primary cyclonic separation device may be used in embodiments of the presently disclosed technology.

Still referring to FIG. 1, at least a portion of the separated catalyst is passed out of the reactor portion 200 and into the catalyst processing portion 300, through catalyst outlet ports 222, 223 via transport lines 506,508, respectively. In some embodiments, additional catalyst may passed via transport line 422 back to the reactor 202 as a recycle stream that is not sent to the catalyst processing portion 300.

As described in the embodiments herein, the catalyst is separated into at least two portions-a first catalyst portion that moves through transport line 506 and a second catalyst portion that moves through transport line 508. The sum of the catalyst flow in transport lines 506 and 508 may be about equal to the catalyst flow in transport line 424 returning the catalyst from the catalyst processing portion 300 to the reactor portion 200. The first catalyst portion moves through transport line 506 to the combustor 350. The second portion of catalyst moves through transport line 508 to an area downstream of the combustor 350, as is described herein.

According to embodiments, the first catalyst portion is transported from the catalyst separation section 210 to the combustor 350. In the combustor 350, the first catalyst portion may be processed by, for example, combustion with oxygen. For example, and without limitation, the catalyst may be de-coked and/or supplemental fuel may be combusted to heat the catalyst. This catalyst is then passed out of the combustor 350 and through the riser 330 to a riser termination separator 378, where the gas and solid components from the riser 330 are at least partially separated. The vapor and remaining solids are transported to a secondary separation device 320 in the catalyst separation section 310 where the remaining catalyst is separated from the gases from the catalyst processing (e.g., gases emitted by combustion of spent catalyst or supplemental fuel, referred to herein as flue gas). The flue gas may pass out of the catalyst processing portion 300 via outlet pipe 432. The separated catalyst (the catalyst of the first catalyst portion) is then passed through the oxygen treatment zone 370 within the catalyst separation section 310.

According to one or more embodiments, the second catalyst portion bypasses the combustor 350 and is mixed with the second catalyst portion (which is processed in the combustor 350). For example, as is depicted in FIG. 1, the second catalyst portion is transported via transfer line 508 into the catalyst processing portion 300 downstream of the combustor 350 and upstream of the oxygen treatment zone 370. At this point, the first catalyst portion and second catalyst portion are recombined, forming a recombined catalyst stream. Following passage through the oxygen treatment zone 370, the combined catalyst stream may be passed to the reactor portion 200 via standpipe 424 and transport riser 430, where it is further utilized in a catalytic reaction. Thus, the catalyst, in operation, may cycle between the reactor portion 200 and the catalyst processing portion 300.

Referring now to the catalyst processing portion 300, as depicted in FIG. 1, the combustor 350 of the catalyst processing portion 300 may include one or more lower reactor portion inlet ports 352 and may be in fluid communication with the riser 330. Oxygen-containing gas, such as air, may be passed through pipe 428 into the combustor 350. The combustor 350 may be in fluid communication with the first catalyst portion via standpipe 506, which may supply the first catalyst portion (e.g., spent catalyst) from the reactor portion 200 to the catalyst processing portion 300 for regeneration. The combustor 350 and riser 330, collectively referred to as the catalyst combustion reactor 302, may operate with similar or identical fluidization regimes as to what was disclosed with respect to the upstream reactor section 250 and downstream reactor section 230 of the reactor portion 200. That is, the combustor 350 may operate as a fluidized bed, such as in a fast fluidized, turbulent, or bubbling bed upflow reactor, while the riser 330 may operate in more of a plug flow manner, such as in a riser reactor. Geometries as described with respect to the upstream reactor section 250 and downstream reactor section 230 may equally apply to the combustor 350 and riser 330. Additionally, the combustor 350 may also include a fuel inlet 354, which may supply a fuel, such as a hydrocarbon stream, to the combustor 350.

As described in one or more embodiments, following separation of flue gas from first catalyst portion in the riser termination separator 378 and secondary separation device 320, treatment of the processed first catalyst portion along with the second catalyst portion (referred to herein as the recombined catalyst stream) with an oxygen-containing gas is conducted in the oxygen treatment zone 370. In some embodiments, the oxygen treatment zone 370 includes a fluid solids contacting device. The fluid solids contacting device may include baffles or grid structures to facilitate contact of the processed catalyst with the oxygen-containing gas. Examples of fluid solid contacting devices are described in further detail in U.S. Pat. Nos. 9,827,543 and 9,815,040. The fluidization regime within the oxygen treatment zone 370 may be bubbling bed type fluidization. The oxygen treatment zone 370 may include an oxygen-containing gas inlet 372, which may supply an oxygen-containing gas to the oxygen treatment zone 370 for oxygen treatment of the recombined catalyst.

In some embodiments, following the oxygen treatment zone 370, the catalyst may be contacted by nitrogen, steam, or another unreactive gas in a strip zone, which may strip one or more components from the catalyst.

As is described herein, in one or more embodiments, it may be beneficial to bypass the combustor 350 with at least a portion of the catalyst. It has been discovered that catalyst may be deactivated in the combustor 350 in respect to dehydrogenation activity. Without being bound by any particular theory, catalyst may be deactivated in the combustor 350 and then reactivated in the oxygen treatment zone 370 by exposure to oxygen. Bypassing the combustor 350 with a portion of the catalyst may result in better catalyst activation, which may be beneficial to dehydrogenation reaction yields and the like.

Additionally, exposure to the supplemental fuel combustion in the combustor 350 may reduce the activity life of the catalyst. That is, permanent deactivation of the catalyst, requiring replacement with new catalyst, may be necessary over long periods of operation, and exposure to supplemental fuel combustion appears to be a main driver of this deactivation. Thus, it is believed that the presently described system, where a portion of the catalyst bypasses the combustor 350, may prolong useful catalyst lifetime, reducing costs by using less new catalyst.

In one or more embodiments, the mass ratio of catalyst flow of the first catalyst portion (to the combustor 350) to the second catalyst portion (bypassing the combustor 350) may be from 3:7 to 9:1, such as from 3:7 to 4:6, from 3:7 to 5:5, from 3:7 to 6:4, from 3:7 to 7:3, from 7:3 to 8:2, from 4:7 to 9:1, from 5:5 to 9:1, from 6:4 to 9:1, from 7:3 to 9:1, or from 8:2 to 9:1. In additional embodiments, the mass ratio of catalyst flow of the first catalyst portion (to the combustor 350) to the second catalyst portion (bypassing the combustor) may be from 3:7 to 7:3, such as from 3:7 to 4:6, from 4:6 to 5:5, from 5:5 to 6:4, from 6:4 to 7:3, or any combination of these ranges. As is described in detail herein, the ratio may be varied based on several factors, such as the thermal needs in the dehydrogenation unit, where the first catalyst portion is heated by combustion of a supplemental fuel while the second portion may not be substantially heated in the catalyst processing portion 300. It is contemplated that a relatively high mass ratio of catalyst flow of the first catalyst portion (to the combustor 350) to the second catalyst portion (bypassing the combustor 350) may be beneficial (e.g., a ratio of at least 3:7 or even greater) because, in these embodiments, the catalyst may be adequately heated by the combustion in the combustor 350 to continuously heat the reactor 202 as is needed for the endothermic dehydrogenation reaction. That is, it is contemplated that a relatively high mass ratio of catalyst flow of the first catalyst portion (to the combustor 350) to the second catalyst portion (bypassing the combustor 350) may be beneficial (e.g., a ratio of at least 3:7 or even greater) because the temperature in the combustor can be operated at a moderate temperature as compared with embodiments where a greater amount of catalyst bypasses the combustor 350, since heat is needed for the endothermic dehydrogenation reaction, which may be mainly supplied by the catalyst or other solids entering the reactor 202. On the other hand, a relatively low mass ratio of catalyst flow of the first catalyst portion (to the combustor 350) to the second catalyst portion (bypassing the combustor 350) may be beneficial (e.g., a ratio of less than or equal to 9:1 or less than or equal to 7:3, or even less) because the second catalyst portion bypassing the combustor 350 is not exposed to the supplemental fuel combustion in the combustor 350, which may cause short term and long term deactivation of the catalyst. That is, in these embodiments, the catalyst that bypasses the combustor may be heated by the catalyst that passes though the combustor 350 while not being exposed to the supplemental fuel combustion that may cause deactivation of the catalyst.

According to additional embodiments, a portion of the catalyst is recycled to the combustor 350. For example, as is shown in FIG. 1, catalyst transfer line 502 may pass catalyst from the catalyst processing portion 300 (upstream of the oxygen treatment zone 370) back to the combustor 350. Such a recycle stream via transfer line 502 may be useful to control the density of catalyst in the combustor 350. For example, when a relatively high amount of catalyst is present in the second catalyst portion (via line 508), the catalyst density in the combustor 350 may be relatedly low, and so recycle of additional catalyst via line 502 may allow for better control of catalyst density to maintain proper fluidization in the combustor 350.

In accordance with some embodiments, a portion of the recombined catalyst stream is recycled to the combustor 350 prior to entering the oxygen treatment zone 370. In such embodiments, transfer line 502 may be downstream of the inlet of transfer line 508 delivering catalyst directly from the reactor portion 200, such that the combining of the first catalyst portion and second catalyst portion occurs upstream of the inlet to transfer line 520. According to additional embodiment, a portion of the first catalyst portion is recycled to the combustor 350 prior to entering the oxygen treatment zone 370. In such embodiments, transfer line 502 may be upstream of the inlet of transfer line 508 delivering catalyst directly from the reactor portion 200, such that only the first catalyst portion is passed into transfer line 502.

In one or more embodiments, the light olefins may be present in a “product stream” sometimes called an “olefin-containing effluent” and include light olefins. Such a stream exits the reactor system of FIG. 1 and may be subsequently processed. As used in the present disclosure, the term “light olefins” refers to one or more of ethylene, propylene, and butene. In additional embodiments, the feed may include ethylbenzene, which is converted to styrene, present in the product stream, which is an embodiments of an olefin-containing material as described herein. The term butene includes any isomers of butene, such as a-butylene, cis-β-butylene, trans-β-butylene, and isobutylene. In some embodiments, the olefin-containing effluent includes at least 30 wt. % light olefins based on the total weight of the olefin-containing effluent. For example, the olefin-containing effluent may include at least 35 wt. % light olefins, at least 45 wt. % light olefins, at least 55 wt. % light olefins, at least 65 wt. % light olefins, or at least 75 wt. % light olefins based on the total weight of the olefin-containing effluent. The olefin-containing effluent may further comprise unreacted components of the feed stream, as well as other reaction products that are not considered light olefins. The light olefins may be separated from unreacted components in subsequent separation steps.

In non-limiting examples, the reactor system 102 described herein may be utilized to produce light olefins from hydrocarbon feed streams. Light olefins may be produced from a variety of hydrocarbon feed streams by utilizing different reaction mechanisms. As described herein, light olefins may be produced by the utilization of dehydrogenation reactions.

According to one or more embodiments, the reaction may be a dehydrogenation reaction. According to such embodiments, the hydrocarbon feed stream may comprise one or more of ethyl benzene, ethane, propane, n-butane, and i-butane. In one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethyl benzene. In one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of propane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of n-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of the sum of ethane, propane, n-butane, and i-butane.

In one or more embodiments, the dehydrogenation reaction may utilize gallium and/or platinum particulate solids as a catalyst. In such embodiments, the particulate solids may comprise a gallium and/or platinum catalyst. As described herein, a gallium and/or platinum catalyst comprises gallium, platinum, or both. The gallium and/or platinum catalyst may be carried by an alumina or alumina silica support, and may optionally comprise potassium. Such gallium and/or platinum catalysts are disclosed in U.S. Pat. No. 8,669,406, which is incorporated herein by reference in its entirety. However, it should be understood that other suitable catalysts may be utilized to perform the dehydrogenation reaction.

In one or more embodiments, the reaction mechanism may be dehydrogenation followed by combustion (in the same chamber). In such embodiments, a dehydrogenation reaction may produce hydrogen as a byproduct, and an oxygen carrier material may contact the hydrogen and promote combustion of the hydrogen, forming water. Examples of such reaction mechanisms, which are contemplated as possible reactions mechanisms for the systems and methods described herein, are disclosed in WO 2020/046978, the teachings of which are incorporated by reference in their entirety herein.

### EXAMPLE

The present example illustrates the variance in dehydrogenation catalytic activity of catalyst which is exposed to combustion of a supplemental fuel. As is described herein, the identification of the result of exposure to combustion of supplemental fuels may provide a rationale for the usefulness of the systems described herein where a portion of catalyst bypasses the combustor.

Propane dehydrogenation testing was conducted at ambient pressure in a lab-scale fixed bed reactor using simulated reaction-combustion-regeneration (standard) cycles and reaction-regeneration (bypass) cycles. For each test, 0.5 g of a catalyst sample was mixed with 1.0 g of inert silicon carbide and loaded into a quartz reactor tube. In a standard cycle, reaction, combustion, and regeneration steps were included. First, a propane dehydrogenation reaction step was performed for 60 seconds with a reactor temperature of 625° C., under a feed composition of 90 mol. % propane and 10 mol. % nitrogen with a total flow rate of 51.38 standard cubic centimeters per minute (sccm) and a weight hourly space velocity (WHSV) of propane of 10 hr⁻¹. Next, the reactor tube was purged under a helium flow and heated to 730° C. Next, a fuel combustion step was performed for 180 seconds with a reactor temperature of 730° C. under a feed composition of 2.5 mol% methane in balance air with a total flow rate of 50 sccm and a WHSV of methane of 0.1hr⁻¹. Next, a regeneration step was performed for 120 seconds with a reactor temperature of 730° C. under a feed composition of 100% air with a flow rate of 40 sccm. Next, the reactor tube was purged under a helium flow and cooled to 625° C. in order to start the next cycle. In a bypass cycle, the steps were performed similarly as in standard cycles except that the fuel combustion step was skipped. That is, after the reaction step and purge under helium, the regeneration step was performed. For each propane dehydrogenation reaction step, the reactor effluent was analyzed using gas chromatography (GC) at 30 seconds time on stream and the propane conversion was calculated from the product gas concentrations on a carbon atom basis. The catalyst utilized for testing contained platinum and gallium on an alumina support.

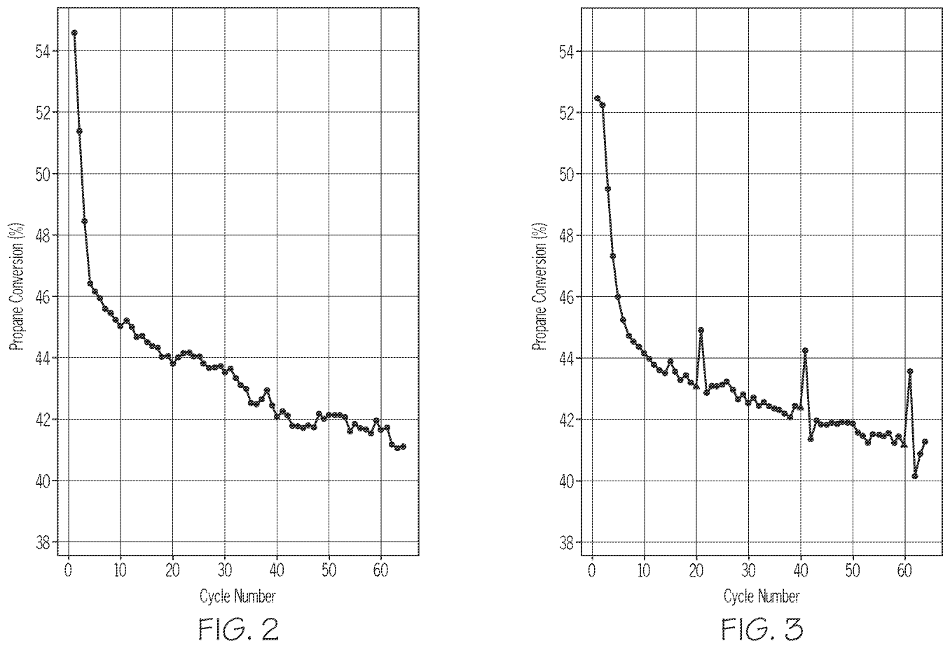

The test was performed with all standard cycles (which include exposure to methane combustion), for which propane conversion data for each subsequent cycle is shown in FIG. 2. As can be seen, the propane conversion steadily declines over the course of the cycles.

Another test was performed with all standard cycles aside from bypass cycles at cycles 20, 40, and 60. Data for this experiment is provided in FIG. 3, which shows a clear increase in propane conversion (indicative of greater dehydrogenation catalytic activity) following the bypass cycles. Thus, it can be analogized that catalyst that bypasses the combustor in the presently disclosed embodiments will generally have greater activity even when both are exposed to a regenerative oxygen treatment.

The present disclosure includes numerous aspects. One aspect is a method for forming a dehydrogenated product, the method comprising: reacting a feed stream in the presence of a catalyst by a dehydrogenation reaction in a reactor to form a dehydrogenated product; separating at least a portion of the product stream from the catalyst; separating the catalyst into at least a first catalyst portion and a second catalyst portion; passing the first catalyst portion to a combustor, wherein the first catalyst portion is heated in the combustor by the combustion of supplemental fuel; passing the first catalyst portion out of the combustor and combining the first catalyst portion with the second catalyst portion downstream of the combustor to form a recombined catalyst stream, such that the second catalyst portion bypasses the combustor; passing the recombined catalyst stream through an oxygen treatment zone; and passing the recombined catalyst stream to the reactor.

Another aspect is any single above aspect or combination of above aspects, wherein the mass ratio of catalyst flow of the first catalyst portion to the second catalyst portion is from 3:7 to 9:1.

Another aspect is any single above aspect or combination of above aspects, wherein the mass ratio of catalyst flow of the first catalyst portion to the second catalyst portion is from 3:7 to 7:3.

Another aspect is any single above aspect or combination of above aspects, wherein the dehydrogenation reaction forms coke on the catalyst.

Another aspect is any single above aspect or combination of above aspects, wherein at least a portion of the coke is combusted in the combustor.

Another aspect is any single above aspect or combination of above aspects, wherein the supplemental fuel comprises hydrogen, methane, ethane, propane, or natural gas.

Another aspect is any single above aspect or combination of above aspects, wherein the product stream comprises one or more of ethylene, propylene, butene, or styrene.

Another aspect is any single above aspect or combination of above aspects, wherein the product stream comprises at least 30 wt. % of ethane, propane, butane, or ethylbenzene.

Another aspect is any single above aspect or combination of above aspects, wherein the fluidization regime in the oxygen treatment zone is bubbling bed type fluidization.

Another aspect is any single above aspect or combination of above aspects, wherein the first catalyst portion is deactivated for dehydrogenation activity in the combustor and then reactivated for dehydrogenation activity in the oxygen treatment zone.

Another aspect is any single above aspect or combination of above aspects, wherein the recombined catalyst stream is passed through a strip zone downstream of the oxygen treatment zone.

Another aspect is any single above aspect or combination of above aspects, wherein the catalyst comprises gallium and platinum.

Another aspect is any single above aspect or combination of above aspects, wherein the catalyst further comprises an alumina support.

Another aspect is any single above aspect or combination of above aspects, wherein the dehydrogenation reaction is followed by combustion of the hydrogen product of the dehydrogenation reaction, and an oxygen carrier material is present in the reactor.

Another aspect is any single above aspect or combination of above aspects, wherein a portion of the recombined catalyst stream or a portion of the first catalyst portion is recycled to the combustor prior to entering the oxygen treatment zone.

In one or more embodiments, streams or the like are described as being “passed” from one system component to another. It should be understood that passing may describe direct passing (where the stream is passed directly between two named system components) or indirect passing (where the stream is passed between two system components through one or more intermediate system components or the line).

It will be apparent to those skilled in the art that various modifications and variations can be made to the presently disclosed technology without departing from the spirit and scope of the technology. Since modifications combinations, sub-combinations and variations of the disclosed embodiments incorporating the spirit and substance of the presently disclosed technology may occur to persons skilled in the art, the technology should be construed to include everything within the scope of the appended claims and their equivalents. Additionally, although some aspects of the present disclosure may be identified herein as preferred or particularly advantageous, it is contemplated that the present disclosure is not limited to these aspects.

It is noted that the various details described in this disclosure should not be taken to imply that these details relate to elements that are essential components of the various embodiments described in this disclosure, even in cases where a particular element is illustrated in each of the drawings that accompany the present description. Unless specifically identified as such, no feature disclosed and described herein should be construed as “essential”. Contemplated embodiments of the present technology include those that include some or all of the features of the appended claims.

In relevant cases, where a composition is described as “comprising” one or more elements, embodiments of that composition “consisting of” or “consisting essentially of” those one or more elements is contemplated herein.

It should be appreciated that compositional ranges of a chemical constituent in a stream or in a reactor should be appreciated as containing, in some embodiments, a mixture of isomers of that constituent. For example, a compositional range specifying butene may include a mixture of various isomers of butene. It should be appreciated that the examples supply compositional ranges for various streams, and that the total amount of isomers of a particular chemical composition can constitute a range.

It is noted that one or more of the following claims and the detailed description utilize the terms “where” or “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that any two quantitative values assigned to a property may constitute a range of that property, and all combinations of ranges formed from all stated quantitative values of a given property are contemplated in this disclosure. Where multiple ranges for a quantitative value are provided, these ranges may be combined to form a broader range, which is contemplated in the embodiments described herein.

As would be understood in the context of the term as used herein, the term “passing” may include directly passing a substance between two portions of the disclosed system and, in some other instances, to mean indirectly passing a substance between two portions of the disclosed system. For example, indirect passing may include steps where the named substance passes through an intermediate separation device, valve, sensor, etc.

1. A method for forming a dehydrogenated product, the method comprising:
reacting a feed stream in the presence of a catalyst by a dehydrogenation reaction in a reactor to form a dehydrogenated product;
separating at least a portion of the product stream from the catalyst;
separating the catalyst into at least a first catalyst portion and a second catalyst portion;
passing the first catalyst portion to a combustor, wherein the first catalyst portion is heated in the combustor by the combustion of supplemental fuel;
passing the first catalyst portion out of the combustor and combining the first catalyst portion with the second catalyst portion downstream of the combustor to form a recombined catalyst stream, such that the second catalyst portion bypasses the combustor;
passing the recombined catalyst stream through an oxygen treatment zone; and
passing the recombined catalyst stream to the reactor.

2. The method of claim 1, wherein the mass ratio of catalyst flow of the first catalyst portion to the second catalyst portion is from 3:7 to 9:1.

3. The method of claim 1, wherein the mass ratio of catalyst flow of the first catalyst portion to the second catalyst portion is from 3:7 to 7:3.

4. The method of claim 1, wherein the dehydrogenation reaction forms coke on the catalyst.

5. The method of claim 4, wherein at least a portion of the coke is combusted in the combustor.

6. The method of claim 1, wherein the supplemental fuel comprises hydrogen, methane, ethane, propane, or natural gas.

7. The method of claim 1, wherein the product stream comprises one or more of ethylene, propylene, butene, or styrene.

8. The method of claim 1, wherein the product stream comprises at least 30 wt. % of ethane, propane, butane, or ethylbenzene.

9. The method of claim 1, wherein the fluidization regime in the oxygen treatment zone is bubbling bed type fluidization.

10. The method of claim 1, wherein the first catalyst portion is deactivated for dehydrogenation activity in the combustor and then reactivated for dehydrogenation activity in the oxygen treatment zone.

11. The method of claim 1, wherein the recombined catalyst stream is passed through a strip zone downstream of the oxygen treatment zone.

12. The method of claim 1, wherein the catalyst comprises gallium and platinum.

13. The method of claim 12, wherein the catalyst further comprises an alumina support.

14. The method of claim 1, wherein the dehydrogenation reaction is followed by combustion of the hydrogen product of the dehydrogenation reaction, and an oxygen carrier material is present in the reactor.

15. The method of claim 1, wherein a portion of the recombined catalyst stream or a portion of the first catalyst portion is recycled to the combustor prior to entering the oxygen treatment zone.
